(19) United States
(12) Patent Application Publication
(10) Pub. No.: US 2012/0232140 A1
(43) Pub. Date: Sep. 13, 2012

(54) PHARMACEUTICAL COMBINATION OF PROSTAGLANDIN COMPOUND AND NSAID FOR THE TREATMENT OF GLAUCOMA AND OCULAR HYPERTENSION

(75) Inventors: Rajesh Kshirsagar, Bangalore (IN); Chandrashekar Kadam, Bangalore (IN); Pravin Kamble, Bangalore (IN); SM Mudda, Bangalore (IN)

(73) Assignee: MICRO LABS LIMITED, Bangalore (IN)

(21) Appl. No.: 13/508,947

(22) PCT Filed: Nov. 1, 2010
(86) PCT No.: PCT/IN10/00717

§ 371 Date: May 9, 2012

(30) Foreign Application Priority Data
Nov. 11, 2009 (IN) 2761/CHE/2009

(51) Int. Cl. A61K 31/235; A61P 27/06; A61P 27/02; A61K 31/20

(52) U.S. Cl. 514/543; 514/559

(57) ABSTRACT

The present invention relates to a pharmaceutical combination comprising a prostaglandin compound and a NSAID. The present invention particularly relates to an ophthalmic composition comprising travoprost and bromfenac for the treatment of glaucoma and ocular hypertension.

### FIELD OF THE INVENTION

The present invention relates to a pharmaceutical combination comprising a prostaglandin compound and a NSAID. The present invention further relates to use of a pharmaceutical combination comprising a prostaglandin compound and a NSAID in the treatment of glaucoma and ocular hypertension. The present invention particularly relates to an ophthalmic composition comprising travoprost and bromfenac for the treatment of glaucoma and ocular hypertension.

### BACKGROUND OF THE INVENTION

The present invention relates to the treatment of glaucoma and ocular hypertension. In particular, the present invention relates to the use of pharmaceutical combination comprising a Prostaglandin compound and a NSAID for the treatment of glaucoma and ocular hypertension.

Prostaglandins (hereinafter, referred to as PGs) are members of class of organic carboxylic acids, which are contained in tissues or organs of human or other mammals, and exhibit a wide range of physiological activity.

In the last decade topically applied prostaglandin compound such as prostaglandin F2α analogues (bimatoprost, latanoprost, travoprost and unoprostone) have become widely used as a means to reduce elevated intraocular pressure in patients with glaucoma and ocular hypertension.

Non-steroidal anti-inflammatory drugs (NSAIDs) are the most frequently prescribed drugs worldwide for the treatment of pain from various etiologies. Various NSAIDs are widely used as ophthalmic solutions such as diclofenac, bromfenac etc. but for different indications such as the treatment of inflammation in patients.

Literature survey does not reveal any pharmaceutical combination of a prostaglandin compound and a NSAID for the treatment of glaucoma and ocular hypertension. Thus there is need in the art for stable formulations comprising combination of a prostaglandin compound and a NSAID for the treatment of glaucoma and ocular hypertension.

### SUMMARY OF THE INVENTION

The object of the present invention is to provide a pharmaceutical combination comprising: a pharmaceutically effective amount of a prostaglandin compound and a pharmaceutically effective amount of a NSAID for the treatment of glaucoma and ocular hypertension.

Another object of the present invention is to provide a topical pharmaceutical composition comprising: a pharmaceutically effective amount of a prostaglandin compound and a pharmaceutically effective amount of a NSAID for the treatment of glaucoma and ocular hypertension.

Yet another object of the present invention is to provide a topical pharmaceutical composition comprising: a pharmaceutically effective amount of travoprost or pharmaceutically acceptable salts thereof and a pharmaceutically effective amount of bromfenac or pharmaceutically acceptable salts thereof for the treatment of glaucoma and ocular hypertension.

Yet another object of the present invention is to provide a process of preparing a topical pharmaceutical composition comprising: a pharmaceutically effective amount of travoprost or pharmaceutically acceptable salts thereof and a pharmaceutically effective amount of bromfenac or pharmaceutically acceptable salts thereof for the treatment of glaucoma and ocular hypertension.

### DETAILED DESCRIPTION OF THE INVENTION

Present inventors have now been surprisingly found that a pharmaceutical composition comprising combination of a prostaglandin compound and a NSAID is useful in treating glaucoma and ocular hypertension. It has further found that a stable ophthalmic composition comprising a prostaglandin and a NSAID can be prepared which can be stable for longer period of time.

Unless indicated otherwise, all ingredient concentrations are presented in units of % weight/volume (% w/v).

Unless indicated otherwise, the term “stabilized” means keeping a formulation clear and antimicrobially effective for its minimum reasonable shelf life, e.g., at least one year.

Unless indicated otherwise, the term “topical pharmaceutical composition” means composition such as ophthalmic compositions or eye drops; nasal drops compositions and the like.

Unless indicated otherwise, the term “NSAID” means an ophthalmologically acceptable non-steroidal anti-inflammatory drug.

The prostaglandins, according to present invention, include all pharmaceutically acceptable prostaglandins, their derivatives and analogues, and their pharmaceutically acceptable esters and salts.

Examples of prostaglandins according to present invention include but not limited to travoprost, latanoprost, bimatoprost, tafluprost and pharmaceutically acceptable salt thereof and the like. The most preferred prostaglandin is travoprost. Preferably travoprost is present 0.004% w/v.

Examples of NSAIDs according to present invention include but not limited to bromfenac, diclofenac, flurbiprofen, ketorolac, nepafenac, amfenac, or indomethacin and pharmaceutically acceptable salt thereof and the like. The most preferred NSAID is bromfenac or pharmaceutically acceptable salt thereof. Preferably bromfenac is present 0.09% w/v.

The compositions of the present invention contain one or more polyethoxylated castor oils.

Examples of polyethoxylated castor oils include but are not limited to commercially available, and include those classified as PEG-2 to PEG-200 castor oils, as well as those classified as PEG-5 to PEG-200 hydrogenated castor oils. Such polyethoxylated castor oils include those manufactured by Rhone-Poulenc (Cranbury, N.J.) under the Alkamuls® brand and those manufactured by BASF (Parsippany, N.J.) under the Cremophor® brand. It is preferred to use the polyethoxylated castor oils classified as PEG-15 to PEG-50 castor oils, and more preferred to use PEG-30 to PEG-35 castor oils. It is most preferred to use those polyethoxylated castor oils known as Cremophor® EL and Alkamuls® EL-620. The most preferred polyethoxylated castor oil is Cremophor® RH-40.

In addition, the compositions of the present invention may contain one or more other ingredients as excipients.

For example, the compositions may include one or more pharmaceutically acceptable buffering agents, preservatives, tonicity-adjusting agents, antioxidants, pH-adjusting agents.

Examples of buffering agents include but are not limited to phosphate, borate, citrate, acetate, carbonate, borate-polyol complexes, boric acid and the like

Examples of preservatives include but are not limited to benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like, chlorhexidine gluconate and the like.

Examples of tonicity-adjusting agents include but are not limited to mannitol, sodium chloride, xylitol, and the like.

Examples of antioxidants, include, but are not limited to, ascorbic acid, malic acid, citric acid, sodium citrate, butylated hydroxyanisole, butylated hydroxytoluene, propyl gallate, sodium ascorbate, sodium metabisulfite and the like and mixtures thereof.

Examples of the alkaline agents that may be used as pH adjusting agents, include, but are not limited to, sodium hydroxide (NaOH), potassium hydroxide (KOH), tromethamine, monoethanolamine, sodium bicarbonate (NaHCO₃) and other organic and inorganic bases.

Examples of the acidic agents that may be used as pH adjusting agents include, but are not limited to, hydrochloric acid, citric acid, tartaric acid, lactic acid and other organic and inorganic acids and the like and mixtures thereof.

Examples of chelating agents include but are not limited to EDTA, sodium edetate, sodium citrate, condensed sodium phosphate and the like.

The various embodiments of the present invention can be assembled in several different ways.

In one embodiment the present invention provides a topical pharmaceutical composition comprising: a pharmaceutically effective amount of a prostaglandin compound and a pharmaceutically effective amount of a NSAID for the treatment of glaucoma and ocular hypertension.

In yet another embodiment the present invention provides a topical pharmaceutical composition comprising: a pharmaceutically effective amount of travoprost or pharmaceutically acceptable salts thereof and a pharmaceutically effective amount of bromfenac or pharmaceutically acceptable salts thereof for the treatment of glaucoma and ocular hypertension.

In yet another embodiment the present invention provides a method for treating glaucoma and ocular hypertension by administering a combination of: travoprost and bromfenac.

In yet another embodiment the present invention provides a topical ophthalmic composition for the treatment of glaucoma and ocular hypertension comprising a pharmaceutically effective amount of a prostaglandin compound and a pharmaceutically effective amount of a NSAID and pharmaceutically acceptable excipients.

In yet another embodiment the present invention provides a method of enhancing the chemical stability of a topical ophthalmic composition comprising combination of a therapeutically-effective amount of prostaglandin compound and a therapeutically-effective amount of NSAID, wherein the method comprises adding a chemically-stabilizing amount of a polyethoxylated castor oil to the composition.

In yet another embodiment the present invention provides a stabilized topical ophthalmic composition for the treatment of glaucoma and ocular hypertension comprising a combination of: travoprost and bromfenac and a chemically-stabilizing amount of a polyethoxylated castor oil wherein polyethoxylated castor oil present from about 2% w/v to about 10% w/v of the composition.

In yet another embodiment the present invention provides a method of enhancing the chemical stability of a topical ophthalmic composition comprising combination of a therapeutically-effective amount of a prostaglandin compound and a therapeutically-effective amount of a NSAID, wherein the method comprises adding a chemically-stabilizing amount of a polyethoxylated castor oil to the composition wherein the composition is packed in LDPE container.

In yet another embodiment the present invention provides a method of enhancing the chemical stability of a topical ophthalmic composition comprising combination of a therapeutically-effective amount of a prostaglandin compound and a therapeutically-effective amount of a NSAID wherein the method comprises adding a chemically-stabilizing amount of a polyethoxylated castor oil to the composition wherein the pH of the composition is from 5 to 9, preferably from 6.8 to 8.8.

In yet another embodiment the present invention provides a method of enhancing the chemical stability of a topical ophthalmic composition comprising combination of a therapeutically-effective amount of a prostaglandin compound and a therapeutically-effective amount of a NSAID, wherein the method comprises adding a chemically-stabilizing amount of a polyethoxylated castor oil to the composition wherein the viscosity of the formulation is from about 2 cps to about 120 cps.

In yet another embodiment the present invention provides a method of enhancing the chemical stability of a topical ophthalmic composition comprising combination of a therapeutically-effective amount of a prostaglandin compound and a therapeutically-effective amount of a NSAID, wherein the method comprises adding a chemically-stabilizing amount of a polyethoxylated castor oil to the composition wherein the composition is stable for more than three months; preferably more than six months, still preferably more than twelve months.

In yet another embodiment the present invention provides a process of preparing a topical ophthalmic composition comprising combination of a therapeutically-effective amount of a prostaglandin compound and a therapeutically-effective amount of a NSAID, wherein the method comprises adding a chemically-stabilizing amount of a polyethoxylated castor oil to the composition wherein polyethoxylated castor oil present from about 2% w/v to about 10% w/v of the composition.

In yet another embodiment the present invention provides a method of enhancing the chemical stability of a topical ophthalmic composition comprising combination of a pharmaceutically effective amount of travoprost or pharmaceutically acceptable salts thereof and a pharmaceutically effective amount of bromfenac or pharmaceutically acceptable salts thereof, wherein the method comprises adding a chemically-stabilizing amount of a polyethoxylated castor oil to the composition wherein the composition is packed in LDPE container.

In yet another embodiment the present invention provides a method of enhancing the chemical stability of a topical ophthalmic composition comprising combination of a pharmaceutically effective amount of travoprost or pharmaceutically acceptable salts thereof and a pharmaceutically effective amount of bromfenac or pharmaceutically acceptable salts thereof wherein the method comprises adding a chemically-stabilizing amount of a polyethoxylated castor oil to the composition wherein the pH of the composition is from 5 to 9, preferably from 6.8 to 8.8.

In yet another embodiment the present invention provides a method of enhancing the chemical stability of a topical ophthalmic composition comprising combination of a pharmaceutically effective amount of travoprost or pharmaceutically acceptable salts thereof and a pharmaceutically effective amount of bromfenac or pharmaceutically acceptable salts thereof, wherein the method comprises adding a chemically-stabilizing amount of a polyethoxylated castor oil to the composition wherein the viscosity of the formulation is from about 2 cps to about 120 cps.

In yet another embodiment the present invention provides a method of enhancing the chemical stability of a topical ophthalmic composition comprising combination of a pharmaceutically effective amount of travoprost or pharmaceutically acceptable salts thereof and a pharmaceutically effective amount of bromfenac or pharmaceutically acceptable salts thereof, wherein the method comprises adding a chemically-stabilizing amount of a polyethoxylated castor oil to the composition wherein the composition is stable for more than three months; preferably more than six months, still preferably more than twelve months.

In yet another embodiment the present invention provides a process of preparing a topical ophthalmic composition comprising a pharmaceutically effective amount of travoprost or pharmaceutically acceptable salts thereof and a pharmaceutically effective amount of bromfenac or pharmaceutically acceptable salts thereof wherein the process comprises step of adding a chemically-stabilizing amount of a polyethoxylated castor oil to the composition.

The invention will be further illustrated by the following examples, which are intended to be illustrative but not limiting.

### Example 1

### Travoprost 0.004% w/v and Bromfenac 0.09% w/v Ophthalmic Solution

TABLE NO. 1

| Sr. No. | Ingredients | Qty/mL | |
|---|---|---|---|
| 1 | Travoprost | 0.04 | mg |
| 2 | Bromfenac Sodium | 1.035 | mg* |
| 3 | Benzalkonium chloride | 0.15 | mg |
| 4 | Boric acid | 3.00 | mg |
| 5 | Disodium edetate | 0.10 | mg |
| 6 | Polyoxyethylene hydrogenated castor | 5.00 | mg |
| | oil 40 (Cremophor RH-40) | | |
| 7 | Tromethamine | 1.20 | mg |
| 8 | Mannitol | 46.00 | mg |
| 9 | Sodium hydroxide | QS to pH | |
| 10 | Water For Injection | QS to 1 | mL |

Parameters

| | |
|---|---|
| Osmolarity (mOsmol/kg) | 291 |
| Viscosity (Cps) | 1.17 |
| Drop size (μl) | 45 |
| pH | 5-9 |

1.035 mg* Bromfenac sodium equivalent to 0.9 mg Bromfenac free acid

### Manufacturing Process:

1) Take Water for injection and purge with nitrogen.
2) Take 30% of WFI add and dissolve Disodium edetate, Benzalkonium chloride, Tromethamine, Boric acid and Mannitol one by one, check the clarity.
3) Take 40% ml of WFI in a beaker, add and dissolve Cremophor RH-40.
4) Weigh the Travoprost in to a glass beaker, add Cremophor solution and stir till the clear solution is obtained.
5) Add and mix the solution of step 2 in to solution of step 4, check the clarity of the solution.
6) Check the pH; if necessary adjust with 10% NaOH.
7) Add and dissolve Bromfenac in solution of step 6.
8) Make up the volume to 100% and check the pH, if necessary adjust with 10% NaOH

The two different batches of formulations as shown in table 1 were taken, one batch was with pH 7 (Batch A) and another was with pH 8 (Batch B).

Both the batches were filled in different containers and studied for stability at different stability conditions. The results obtained are presented for Batch A in Table 2 & 3 and for Batch B in Table 4 & 5

TABLE NO. 2
Assay

| | | | Stability Condition | | | |
|---|---|---|---|---|---|---|
| | | | Batch A | | | |
| | | | 40° C. ± 2° C./ | | | |
| Sr. | | | 25% RH ± 5% RH | | 60° c. | |
| No. | Tests | Initial | 1 Month | 2 Months | 1 Week | 2 Weeks |
| 1 | Travoprost | 97.5 | 95.5 | 94.2 | 96.3 | 85.3 |
| | (%) | | | | | |
| | Bromfenac | 95.2 | 86.8 | 85.2 | 85.2 | 76.9 |
| | (%) | | | | | |
| 2 | pH | 7 | 6.89 | 6.84 | 7.00 | 6.96 |

Pack: 5 ml BFS

TABLE NO. 3

| | | | Stability Condition | | |
|---|---|---|---|---|---|
| | | | Batch A | | |
| | | | | | 25° C. ± 2° C./ |
| | | | 30° C. ± 2° C./ | | 40% RH ± |
| Sr. | | | 65% RH ± 5% RH | | 5% RH |
| No. | Tests | Initial | 1 Month | 3 Months | 1 Month |

Assay

| | | | | | |
|---|---|---|---|---|---|
| 1 | Travoprost (%) | 97.5 | 98.3 | 96.5 | 93.50 |
| | Bromfenac (%) | 95.2 | 86.5 | 84.2 | 84.53 |
| 2 | pH | 7 | 6.99 | 6.48 | 7.03 |

Pack: 5 ml BFS

TABLE NO. 4

| | | | Stability Condition | | |
|---|---|---|---|---|---|
| | | | Batch B | | |
| | | | 40° C. ± 2° C./ | | |
| Sr. | | | 25% RH ± 5% RH | 60° c. | |
| No. | Tests | Initial | 1 Month | 1 Week | 2 Weeks |

Assay

| | | | | | |
|---|---|---|---|---|---|
| 1 | Travoprost (%) | 99.3 | 98.3 | 101.1 | 95.3 |
| | Bromfenac (%) | 96.8 | 95.0 | 95.1 | 92.0 |
| 2 | pH | 8 | 7.79 | 7.92 | 7.80 |

Pack: 5 ml BFS

TABLE NO. 5

| | | | Stability Condition | |
|---|---|---|---|---|
| | | | Batch B | |
| | | | 30° C. ± 2° C./ | 25° C. ± 2° C./ |
| Sr. | | | 65% RH ± 5% RH | 40% RH ± 5% RH |
| No. | Tests | Initial | 1 Month | 1 Month |

Assay

| | | | | |
|---|---|---|---|---|
| 1 | Travoprost (%) | 99.3 | 98.8 | 99.5 |
| | Bromfenac (%) | 96.8 | 96.8 | 98.1 |
| 2 | pH | 8 | 7.84 | 7.91 |

Pack: 5 ml BFS

1. A topical pharmaceutical composition comprising: a pharmaceutically effective amount of a prostaglandin compound and a pharmaceutically effective amount of a NSAID and one or more pharmaceutically acceptable excipients for the treatment of glaucoma and ocular hypertension.

2. A method for treating glaucoma and ocular hypertension by administering a composition according to claim 1.

3. A topical pharmaceutical composition according to claim 1 wherein the pH of the composition is from 6.8 to 8.8.

4. A topical pharmaceutical composition according to claim 1 wherein the viscosity of the formulation is from about 2 cps to about 120 cps.

5. A topical pharmaceutical composition according to claim 1 wherein the composition is packed in LDPE container.

6. A topical ophthalmic composition comprising: a pharmaceutically effective amount of travoprost or pharmaceutically acceptable salts thereof and a pharmaceutically effective amount of bromfenac or pharmaceutically acceptable salts thereof and one or more pharmaceutically acceptable excipients for the treatment of glaucoma and ocular hypertension.

7. A method for treating glaucoma and ocular hypertension by administering a composition according to claim 6.

8. A topical ophthalmic composition comprising: a pharmaceutically effective amount of travoprost or pharmaceutically acceptable salts thereof and a pharmaceutically effective amount of bromfenac or pharmaceutically acceptable salts thereof and polyethoxylated castor oil for the treatment of glaucoma and ocular hypertension.

9. A method for treating glaucoma and ocular hypertension by administering a composition according to claim 8.

10. A topical ophthalmic composition according to claim 6 wherein the pH of the composition is from 6.8 to 8.8.

11. A topical ophthalmic composition according to claim 6 wherein the viscosity of the formulation is from about 2 cps to about 120 cps.

12. A topical ophthalmic composition according to claim 6 wherein polyethoxylated castor oil present from about 2% w/v to about 10% w/v of the composition.

13. A topical ophthalmic composition comprising:
a) Travoprost 0.004% w/v
b) Bromfenac 0.09% w/v
c) Polyethoxylated castor oil from about 2% w/v to about 10% w/v
d) One or more pharmaceutically acceptable excipients.

14. A method for treating glaucoma and ocular hypertension by administering a composition according to claim 13.

15. A process of preparing a topical ophthalmic composition comprising a pharmaceutically effective amount of travoprost or pharmaceutically acceptable salts thereof and a pharmaceutically effective amount of bromfenac or pharmaceutically acceptable salts thereof wherein the process comprises step of adding a chemically-stabilizing amount of a polyethoxylated castor oil to the composition.
